(19) United States
(12) Patent Application Publication
(10) Pub. No.: US 2006/0047285 A1
(43) Pub. Date: Mar. 2, 2006

(54) Insertion device for a body lumen

(76) Inventor: Betsy Charlotte Fields, San Francisco, CA (US)

Correspondence Address: Edward J. Lynch;Duane Morris LLP, 20th Floor, One Market, Spear Tower, San Francisco, CA, 94105, US

(21) Appl. No.: 11/212,516

(22) Filed: Aug. 24, 2005

Related U.S. Application Data
(60) Provisional application No. 60/604,779, filed on Aug. 26, 2004.

(51) Int. Cl. A61F 11/00

(52) U.S. Cl. 606/108

(57) ABSTRACT

Device are provided for guided placement of an elongated element within a body lumen. A device embodying features of the invention may include a shield, preferably dish shaped, having a distal face configured to engage the opening of the body lumen. A passage way extends through the shield. A guide tube is secured to the shield and extends distally from the distal face of the shield. The guide tube is aligned with the opening of the shield and has an inner lumen configured to guide an elongated element through the tube and to discharge the element into the body lumen.

### RELATED APPLICATIONS

This application is related to, and hereby claims the benefit of U.S. provisional application No. 60/604,779, filed on Aug. 26, 2004, entitled INSERTION DEVICE. Priority is based on this application and it is incorporated herein in its entirety.

### FIELD OF THE INVENTION

This invention is generally directed to guide devices to facilitate insertion of an elongated element into a body lumen. In particular, the invention relates to devices for the insertion of tampons, suppositories and the like into a female's vaginal canal.

### BACKGROUND OF THE INVENTION

Insertion of elongated elements into a body lumen, such as insertion of tampons, suppositories and the like into the vaginal canal, can often be difficult for inexperienced users. Thus, there is need for a device to guide placement of elongated elements into a body lumen which allows for effective and comfortable delivery of the element.

### SUMMARY OF THE INVENTION

This invention is generally directed to insertion devices to facilitate insertion of an elongated element into a body lumen. In particular, the invention relates to devices for the insertion of tampons, suppositories and the like into a female's vaginal canal.

The guide device generally comprises a shield, preferably dish shaped, having a distal face configured to engage the opening of the body lumen. An opening extends through the shield. A guide tube is secured to the shield and extends distally from the distal face of the shield. The guide tube is aligned with the opening of the shield and has an inner lumen configured to guide an elongated element through the tube and to discharge the element into the body lumen.

The device can help inexperienced users to learn how to insert elongated elements such as tampons, suppositories and the like. The device also allows for the elongated element to be delivered effectively and comfortably. These and other advantages of the present invention are described in more detail in the following written description and the accompanying drawings.

### BRIEF DESCRIPTION OF THE DRAWINGS

FIG. 1 is a cross sectional view of a body lumen with an embodiment of the device mounted on a user's finger and positioned at the opening of the body lumen including a guide tube inserted within the body lumen.

FIG. 2 is a cross sectional view of a body lumen with an embodiment of the device mounted on the user's finger and positioned at the opening of the body lumen including a guide tube inserted within the opening and an elongated element extending past the distal end of the tube.

FIG. 3 is a side elevational view of a device embodying features of the invention.

FIG. 4 is a plan view of a device embodying features of the invention.

FIG. 5 is a longitudinal cross sectional view of a device embodying features of the invention taken along lines 5-5 in FIG. 4.

FIG. 6 is a bottom plan view of a device embodying features of the invention.

FIG. 7 is a transverse cross sectional view of the device taken lines 7-7 in FIG. 5.

FIG. 8 is a transverse cross sectional view of the device taken along lines 8-8 in FIG. 5.

### DETAILED DESCRIPTION OF THE INVENTION

The present invention provides devices to facilitate the insertion of an elongated element 12 into a body lumen 16. For example, devices and methods having features of the invention may be used to facilitate insertion of tampons, suppositories and the like into a female's vaginal canal.

The insertion device 18 generally comprises a shield 20, preferably dish shaped, having a distal face 22 configured to engage the opening of the body lumen 16. An opening 24 extends through the shield 20. A guide tube 28 is secured to the shield and extends distally from the distal face 22 of the shield 20. The guide tube 28 is aligned with the opening 24 of the shield 20 and has an inner lumen configured to guide an elongated element 12 through the tube 28 and to discharge the element 12 into the body lumen 16. Preferably, the insertion device 18 has a finger grip 32 on the proximal surface 36 of the shield 20 to allow the user to manually grip the device and properly position the shield 20 adjacent the opening of the body lumen 16. The finger grip 32 may be a ring secured to the shield 20 and configured to slip over the user's finger, allowing for easy handling.

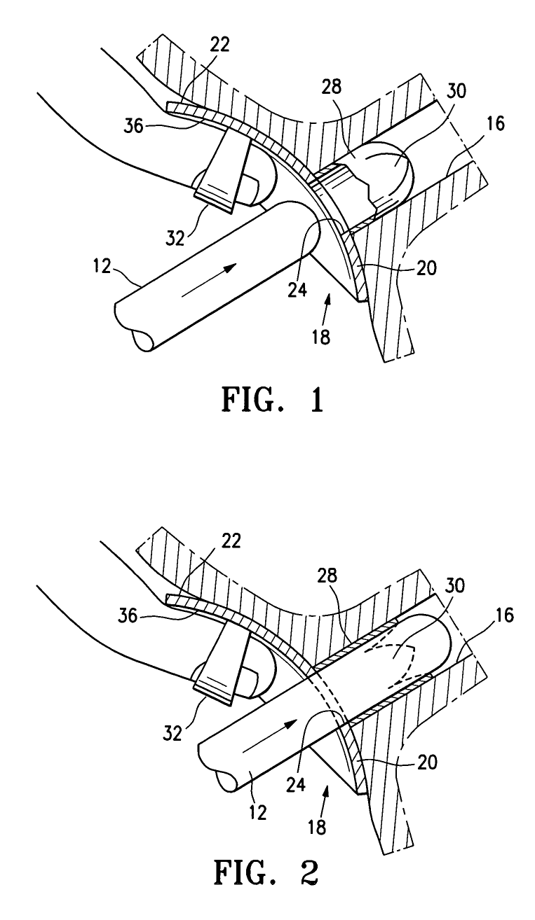

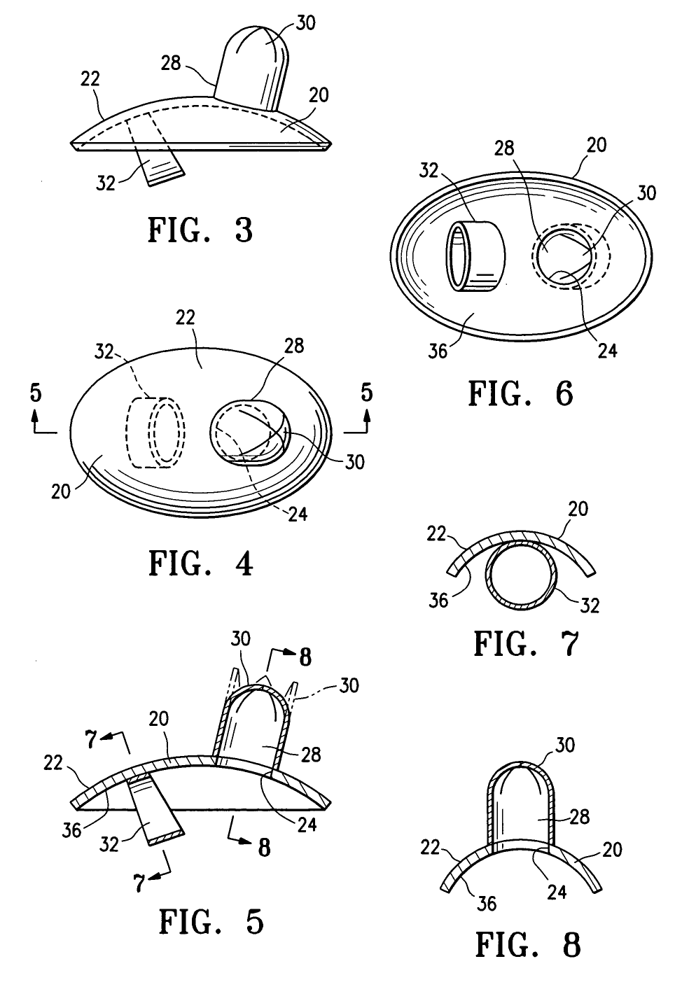

A device embodying features of the invention shown in FIGS. 1-8 features a curved, oval shaped shield 20 made of thermoformed plastic such as PETG. The embodiment in FIGS. 1 and 2 show a shield which is approximately 6 cm long and 3.5 cm wide across the widest portion of the shield, and about 1 cm deep at the deepest part. The shield may come in various sizes and the dimensions may be altered to accommodate differences among users. Preferably, the shield 20 is clear to allow for maximum visualization.

The shield 20 has an opening 24, preferably about 1 cm in diameter, which is located off-set from a central axis on the vertical axis of the shield, preferably about 1 cm from the central axis of the shield. The guide tube 28 is secured to the shield 22 and is aligned with the opening 24. Both the guide tube 28 and the opening 24 are preferably configured to slidably guide the elongated element 12 through the opening 24 of the guide tube 28 into a body lumen 16 such as a female's vaginal canal.

The guide tube 28 is configured to guide an elongated element through the inner lumen of the guide tube into the body lumen 16. The distal end of the guide tube may be petalled to avoid traumatic engagement with the patient's tissue surrounding the opening of the body lumen and within the body lumen 16. In the embodiment shown in FIGS. 1-8 the distal end of the guide tube 28 has four petals 40 similar to the distal end of current tampon applicators, however, the device may contain three or more petals. Preferably the guide tube is about 2.5 cm long for intravaginal insertion of elongated elements. The dimensions of the device may be altered to provide comfort and facilitate use of the device.

The guide tube 28 is generally at an angle with respect to the distal surface of the shield 20. The angle of the tube 28 may be about 30° to about 130°, and for intravaginal use is inclined away from the central axis of the device at an angle of about 45°0 to about 90°, preferably about 60° to about 70° with respect to the perpendicular line passing through the shield. The angle may be varied for comfort and ease of insertion.

The device 18 may come in different sizes to accommodate various elongated elements, e.g. one size that fits slim tampons, one size that fits regular tampons, and one size that fits super absorbency tampons, which are thicker. Thus, the device could come in different forms scaled up or down depending on the size of the elongated element to be inserted.

The proximal face 36 of the shield may include a finger grip 32. The finger grip 32 is a ring of suitable material such as plastic 1 cm in diameter, and may be formed of the same plastic material as the guide tube 28, preferably polypropylene. A more compliant plastic may be used to more comfortably adjust the size of the ring to accommodate different finger sizes, for example if one size of the device was intended to work for all or most users. The center of the finger grip 32 may be located on the vertical axis of the shield about 1.5 cm from the central axis of the shield 20.

While particular forms of the invention have been illustrated and described herein, it will be apparent that various modifications and improvements can be made to the invention. Moreover, individual features of embodiments of the invention may be shown in some drawings and not in others, but those skilled in the art will recognize that individual features of one embodiment of the invention can be combined with any or all the features of another embodiment. Accordingly, it is not intended that the invention be limited to the specific embodiments illustrated. It is therefore intended that this invention be defined by the scope of the appended claims as broadly as the prior art will permit.

Terms such as “element”, “member”, “device”, “sections”, “portion”, “section”, “steps” and words of similar import when used herein shall not be construed as invoking the provisions of 35 U.S.C §112(6) unless the following claims expressly use the terms “means” or “step” followed by a particular function without specific structure or action. All patents and all patent applications referred to above are hereby incorporated by reference in their entirety.

What is claimed is:

1. A device to facilitate insertion of an elongated element into a body lumen through an opening of the body lumen, comprising:
a. a shield having a distal face configured to engage the opening of the body lumen; and
b. a guide tube which is secured to the shield, which extends distally from the distal face of the shield and which has an inner lumen which is configured to guide an elongated element through the tubular guide and to discharge the element into the body lumen.

2. The device of claim 1 wherein the inner lumen of the guide tube is in alignment with the body lumen.

3. The device of claim 1 wherein the guide tube is at an angle with respect to a line perpendicular to the distal face of the shield.

4. The device of claim 1 wherein the shield has an opening which is in alignment with the inner lumen of the guide tube.

5. The device of claim 3 wherein the guide tube is inclined toward a center line axis.

6. The device of claim 3 wherein the angle with respect to a line perpendicular to the distal face of the shield is about 30° to about 130°.

7. The device of claim 3 wherein the angle with respect to a line perpendicular to the distal face of the shield is about 45° to about 90°.

8. The device of claim 3 wherein the angle with respect to a line perpendicular to the distal face of the shield is about 60° to about 70°.

9. The device of claim 1 wherein the distal tip of the guide tube is petalled.

10. The device of claim 9 wherein the distal tip of the guide tube has a plurality of petals.

11. The device of claim 10 wherein the distal tip of the guide tube has four petals.

12. The device of claim 1 wherein a finger grip is provided on the proximal face of the shield.

13. The device of claim 12 wherein the finger grip is configured to slip over a person's finger.

14. The device of claim 1 wherein the finger grip is formed of plastic.

15. The device of claim 1 wherein the guide tube is off set from the center line axis of the shield.

16. The device of claim 15 wherein the finger grip is off set from the guide tube and on the opposite side of the center line axis of the shield.

17. The device of claim 1 wherein the shield is formed of plastic.

18. The device of claim 17 wherein the shield is formed of thermoformed PETG.

19. The device of claim 1 wherein the shield is clear.

20. The device of claim 1 wherein the shield is about 6 cm tall.

21. The device of claim 1 wherein the shield is about 3.5 cm wide.

22. The device of claim 1 wherein the shield is 1 cm deep at the deepest part of the shield.

23. The device of claim 1 wherein the guide tube is configured to guide a tampon into a body cavity.

24. The device of claim 1 wherein the guide tube is configured to guide a suppository into a body cavity.

25. A device to facilitate insertion of an elongated element into a female's vaginal canal, comprising:
a. a shield having a distal face configured to engage the female's external genitalia; and
b. a guide tube which extends distally from the distal face of the shield, which has a distal end configured for insertion into the female's vaginal canal and which has an inner lumen configured to guide an elongated element through the guide tube and to discharge the elongated element into the female's vaginal canal.

26. The device of claim 25 wherein the inner lumen of the guide tube is in alignment with the female's vaginal canal.

27. The device of claim 26 wherein the guide tube is an angle with respect to a line perpendicular to the distal face of the shield.

28. The device of claim 25 wherein the shield has an opening which is in alignment with the inner lumen of the guide tube.

29. The device of claim 27 wherein the guide tube is inclined toward a center line axis.

30. The device of claim 27 wherein the angle with respect to a line perpendicular to the distal face of the shield is about 30° to about 130°.

31. The device of claim 27 wherein the angle with respect to a line perpendicular to the distal face of the shield is about 45° to about 90°.

32. The device of claim 27 wherein the angle with respect to a line perpendicular to the distal face of the shield is about 60° to about 70°.

33. The device of claim 25 wherein the distal tip of the guide tube is petalled.

34. The device of claim 33 wherein the distal tip of the guide tube has a plurality of petals.

35. The device of claim 34 wherein the distal tip of the guide tube has four petals.

36. The device of claim 25 wherein the guide tube is off set from the center line axis of the shield.

37. The device of claim 25 wherein a finger grip is provided on the proximal face of the shield.

38. The device of claim 37 wherein the finger grip is off set from the center line axis and on the opposite side of the center line axis from the guide tube.

39. The device of claim 37 wherein the finger grip is a ring secured to the shield which is configured to slip over a person's finger.

40. The device of claim 37 wherein the finger grip is formed of plastic.

41. The device of claim 25 wherein the guide tube is configured to guide a tampon into a female's vaginal canal.

42. The device of claim 25 wherein the guide tube is configured to guide a suppository into a female's vaginal canal.

43. A method for facilitating insertion of an elongated element into a body lumen, comprising:
a. providing a device having a shield with a distal face configured to engage the opening of the body lumen and a guide tube which is secured to the shield and which extends distally from the distal face of the shield and which has an inner lumen which is configured to guide an elongated element through the tubular guide and to discharge the element into the body lumen;
b. inserting the guide tube of the device into the body lumen;
c. inserting the elongated element though the guide tube; and
d. discharging the element into the body lumen.

44. The method of claim 43 wherein the inner lumen of the guide tube is in alignment with the body lumen.

45. The method of claim 43 wherein the guide tube is at an angle with respect to a line perpendicular to the distal face of the shield.

46. The method of claim 43 wherein the shield has an opening which is in alignment with the inner lumen of the guide tube.

47. The method of claim 45 wherein the guide tube is inclined toward a center line axis.

48. The method of claim 45 wherein the angle with respect to a line perpendicular to the distal face of the shield is about 30° to about 130°.

49. The method of claim 45 wherein the angle with respect to a line perpendicular to the distal face of the shield is about 45° to about 90°.

50. The method of claim 45 wherein the angle with respect to a line perpendicular to the distal face of the shield is about 60° to about 70°.

51. The method of claim 43 wherein the distal tip of the guide tube is petalled.

52. The method of claim 51 wherein the distal tip of the guide tube has a plurality of petals.

53. The method of claim 52 wherein the distal tip of the guide tube has four petals.

54. The method of claim 43 wherein a finger grip is provided on the proximal face of the shield.

55. The method of claim 54 wherein the finger grip is configured to slip over a person's finger.

56. The method of claim 43 wherein the finger grip is formed of plastic.

57. The method of claim 43 wherein the guide tube is off set from the center line axis of the shield.

58. The method of claim 57 wherein the finger grip is off set from the guide tube and on the opposite side of the center line axis of the shield.

59. The method of claim 43 wherein the shield is formed of plastic.

60. The method of claim 59 wherein the shield is formed of thermoformed PETG.

61. The method of claim 43 wherein the shield is clear.

62. The method of claim 43 wherein the shield is about 6 cm tall.

63. The method of claim 43 wherein the shield is about 3.5 cm wide.

64. The method of claim 43 wherein the shield is 1 cm deep at the deepest part of the shield.

65. The method of claim 43 wherein the guide tube is configured to guide a tampon into a body cavity.

66. The method of claim 43 wherein the guide tube is configured to guide a suppository into a body cavity.
